(19) United States
(12) Patent Application Publication
(10) Pub. No.: US 2017/0001194 A1
(43) Pub. Date: Jan. 5, 2017

(54) MICROFLUIDIC DEVICE, SYSTEM, AND METHOD

(71) Applicant: KONINKLIJKE PHILIPS N.V., Eindhoven (NL)

(72) Inventors: WILLEM-JAN AREND DE WIJS, EINDHOVEN (NL); MENNO WILLEM JOSE PRINS, EINDHOVEN (NL)

(73) Assignee: Koninklijke Philips N.V.

(21) Appl. No.: 15/106,623

(22) PCT Filed: Dec. 16, 2014
(86) PCT No.: PCT/EP2014/077853

§ 371 Date: Jun. 20, 2016

(30) Foreign Application Priority Data
Dec. 23, 2013 (EP) 13199272.9

(51) Int. Cl. B01L 3/00; C12N 15/10

(52) U.S. Cl. CPC B01L 3/50273; C12N 15/1013; B01L 3/502761; B01L 3/502738; B01L 2200/0647; B01L 2400/043

(57) ABSTRACT

The present invention relates to a micro-fluidic device for use in a micro-fluidic system. A rigid base structure is provided with a flexible membrane. An external magnetic driver moves from a first position to a second position underneath the micro-fluidic device whilst applying a magnetic field. A droplet containing magnetic particles will be attracted to the external magnetic driver. The flexible membrane is thin, and therefore the micro-fluidic device can be brought closer to the external magnetic driver, thus increasing the magnetic force incident on the fluid drop. A force will be exerted on the flexible membrane, so deflecting the flexible membrane, thus bringing the droplet containing magnetic particles closer to the external magnetic driver. The effect of the increased magnetic field is to increase the packing density of the magnetic droplet. Therefore, a droplet with higher integrity, and less susceptible to splitting, may be moved through the micro-fluidic device.

This invention was made with US Government support under HR0011-12-C-0007 awarded by the Defense Advanced Research Projects Agency. The US Government has certain rights in this invention.

### FIELD OF THE INVENTION

The present invention relates to a micro-fluidic device for fluidic sample analysis. In particular, the invention relates to a micro-fluidic device for transferring fluid containing a plurality of magnetic particles, a testing device, to a method for controlling fluid flow, and to a micro-fluidic system.

### BACKGROUND OF THE INVENTION

A trend in clinical diagnostics is towards point-of-care solutions or integrated bench-top systems. This means that diagnostic tests need to be performed closer to the patient and/or in a decentralized system, in a much shorter time scale. Ease of use is also an important characteristic of point-of-care diagnostics or of an industrial or laboratory or clinical use, because the tests might be performed by a patient (for point-of-care) and the tests can be less expansive (industrial, laboratories or clinical use). One specific feature that is important is the ability for a user to insert a sample into an analyzer simply, and to obtain a result quickly. Sample preparation often involves sample volumes in the millilitre, microlitre, or nanolitre range. Therefore, samples must be prepared carefully so that reagents or analytes are not wasted.

In some types of such systems, the analysis involves the use of magnetic particles that are suspended in a liquid, which may be driven (e.g. for mixing samples or for capturing targets in the sample for further analysis) by a magnetic source. In those systems or micro-fluidic device, it may further be useful to move the magnetic particles from a first microfluidic element (e.g. container, compartment, chamber, channel) to a second microfluidic element without necessarily moving all the fluid from the first to the second microfluidic elements this can be useful for example to drive magnetic particles in different stages of a micro-fluidic process, such as in DNA purification.

WO 2009/083862 discloses a valve-like structure between said two microfluidic elements, using magnetic driver to drag the particles across the valve-like structure from the first to the second microfluidic elements.

### SUMMARY OF THE INVENTION

There may, thus, be a need to provide enhanced means for controllably transferring a fluid containing a plurality of magnetic particles inside a micro-fluidic transition path.

The object of the present invention is solved by the subject-matter of the independent claims, wherein further embodiments are incorporated in the dependent claims.

It should be noted that the following described aspects of the invention apply also to a micro-fluidic system, and a method of controlling fluid flow.

According to the present invention, a micro-fluidic device for fluidic sample analysis is provided and arranged to be positioned in a micro-fluidic controller comprising an external magnetic driver. The micro-fluidic device comprises:
a (e.g. rigid) base structure;
a flexible membrane (e.g. a foil); and
a micro-fluidic transition path limited by, at a bottom side, at least a portion of the base structure and by, at a top side, at least a portion of the flexible membrane, and extending between at least one inlet to be in communication with a first region and at least one outlet to be in communication with a second region.

The micro-fluidic transition path may be provided in the base structure or defined between the base structure and the flexible membrane. The micro-fluidic transition path is typically a transition path between said first microfluidic element and said second microfluidic element. This transition path can allow, under certain conditions, a part of the fluids and/or elements included in this fluid to move from the first element to the second element, and acts therefore as a valve between the first and the second microfluidic elements.

The microfluidic device is further adapted such that, once in position in the microfluidic controller, the flexible membrane is placed in proximity to said external magnetic driver. The micro-fluidic device is further configured so that when a fluid containing a plurality of magnetic particles approaches the micro-fluidic transition path, and a magnetic force is applied by the external magnetic driver near the flexible membrane, at least a part of the magnetic particles are moved away from the base structure towards the flexible membrane. The flexible membrane being deflectable away from the base structure, at least a part of the magnetic particles can be moved towards and located, when the flexible membrane is deflected, and under the action of the external magnetic driver, a region beyond the in rest position of the flexible membrane. The in rest position of the flexible membrane is the position at which the flexible membrane is not deflected under a mechanical force applied to it. The magnetic particles can move without the fluid or with a part of the fluid, depending on the configuration of the system.

Advantageously, because the surface of the micro-fluidic device covering the micro-fluidic transition path is flexible, it is thin in comparison with the rest of the base structure, enabling the flexible membrane, and hence the fluid droplet, to be placed closer to an external magnetic driver.

Therefore, when the fluid containing magnetic particles is on the flexible membrane, and the external magnetic source applies a magnetic field to the fluid, the proximity of the magnetic particles in the fluid and the external magnetic driver may cause a magnetic force converted into a mechanical force to be exerted on the flexible membrane.

Furthermore the improvement related to the proximity between the magnetic particles in the fluid and the external magnetic driver in-turn increases the magnetic field gradient and the absolute magnetic field that the magnetic particles experience. As a consequence, the attraction force between the particles and the magnetic driver is increased, resulting in a higher packing density of magnetic particles inside the fluid droplet, and a higher attraction strength, even when and if the external magnetic driver is moved. The magnetic particles are thus better driven along the transition path, which improves the efficiency and reliability of the valve-like function of the transition path.

This deflection allows a proximity between the flexible membrane and the external magnetic driver to less than 100 micrometers.

Furthermore, since the valve-like function of the transition path is improved, the alignment tolerance required between the micro-fluidic device and the microfluidic controller can be greater, giving rise to a more reliable and/or more robust, less weak system.

According to the invention, a micro-fluidic system is provided. The system comprises:
a micro-fluidic controller, comprising:
a micro-fluidic device placement area compatible with a micro-fluidic device holder;
a magnetic driver configured to apply a magnetic field to the micro-fluidic device placement area; and
said micro-fluidic device.

According to the invention, a fluidic medium can be introduced into the micro-fluidic device, and the micro-fluidic device is secured in the micro-fluidic device placement area of the microfluidic controller. When the plurality of magnetic particles approaches a micro-fluidic transition path of the micro-fluidic device, and a magnetic force is applied by the magnetic driver near the flexible membrane of the micro-fluidic controller, the magnetic particles are attracted towards the magnetic driver. This may possibly cause a force to be exerted onto the flexible membrane, without or with at least a part of the fluid. The flexible membrane is deflectable in the direction of the magnetic driver. This causes the magnetic particles to move towards the magnetic driver, optionally with a part of the fluid.

Also according to the invention, a testing device is provided. The testing device comprises:
at least two fluidic elements;
said micro-fluidic device; and
a magnetic particle transferrer located underneath the micro-fluidic device;
wherein the at least two fluidic elements are connected through the micro-fluidic device via the micro-fluidic transition path, and, in use, the magnetic particle transferrer moves a quantity of magnetic particles, optionally with no fluid or with only a part of fluid, from a first to a second of the at least two fluidic elements.

Also according to the invention, a method is provided for controlling fluid flow. The method comprises the steps of:
a) inserting fluid containing a plurality of magnetic particles into a micro-fluidic device or inserting fluid into a micro-fluidic device containing magnetic particles arranged to be in contact with the fluid, the micro-fluidic device comprising a flexible membrane covering a micro-fluidic transition path; and
b) applying a magnetic field to the micro-fluidic device so as to cause the magnetic particles to be attracted towards the flexible membrane,
c) deflecting the flexible membrane in the direction of the motion of the magnetic particles, causing at least a part of the magnetic particles to be located, with the fluid, beyond the in rest position of the flexible membrane.
Steps b) and c) may be implemented simultaneously.
Step c) may be at least partly caused by the motion of the magnetic particles, attracted towards the flexible membrane according to step b), with or without a part of the fluid exerting a fluidic force onto the flexible membrane.

The flexible membrane can be made from a thin material, and may be for example a foil, which significantly reduces the absolute distance between the nearest magnetic particles in the droplet of fluid, and the magnetic driver, relative to the situation when this flexible membrane is replaced by a thicker or more rigid structure, e.g. a structure having a thickness greater than 100 micrometres.

Advantageously, this enables a significant improvement of proximity between the plurality of magnetic particles in the fluid and the magnetic driver. This in turn increases both the magnetic field gradient that the magnetic particles experience, as well as the absolute magnetic field strength. The higher gradient and field strength increases the attraction force of the magnetic driver to the particles, and results in a higher packing density of the magnetic particles, and a higher attraction strength when and if the magnetic driver is moved. Thus, it is for example less likely that the droplets of fluid containing the magnetic particles will split into several droplets whilst moving through the micro-fluidic valve (i.e. the microfluidic transition path).

In this application, the term “packing density” refers to the density of magnetic particles relating to the inverse of the average distance separating magnetic particles in a fluid. When a magnetic force is applied to a fluid containing magnetic particles, magnetic forces between each magnetic particle causes a decrease in the average separation of the particles, thus increasing the packing density.

In this application, the term “micro-fluidic transition path” means a microfluidic path between and in communication with at least a first region or microfluidic element and at least a second region or microfluidic element, and having specific microfluidic properties with respect to said first and second regions. Preferably, such microfluidic properties (which might include hydrophobicity with respect to hydrophilicity of first and/or second regions) are such that the microfluidic transition path has a valve-like function, preventing at least a part of a fluid contained in the first region to go to the second region, and allowing at least a part of the magnetic particles to come across the microfluidic transition path, without or with at least a part of the fluid, once the process according to the invention is implemented (i.e. by using at least a magnetic driver). Micro-fluidic transition path can also be considered as a channel of the micro-fluidic device extending between compartments in which some fluids are confined to a certain area. The geometry of such channels or compartments can adopt many suitable forms. For instance circular or rectangular areas in which samples are collected for further processing, and linear channels connecting the aforementioned areas, could be considered micro-fluidic transition paths. The micro-fluidic transition path may be provided in a substrate material by various methods known to the skilled person, such as edging, milling, embossing, moulding, printing, and the like.

Alternatively, the channels can be present in the form of areas with surface properties that differ from the surrounding surface of the substrate in such a way that the fluids remain confined within or outside the channels. For example, such channels can be produced from glass surfaces, which are functionalized with a hydrophobic layer of silane. These layers can then be etched with a mask in order to obtain the micro-fluidic channels.

In this application, the term “a flexible membrane” is to be understood to mean a membrane which can be more easily deflected with respect to the base structure under a similar mechanical force applied perpendicularly to their respective main surfaces, and which (i) can be deflected by using an external mechanical force exerted by an underpressure or an overpressure created by gas or by another fluid flowed by e.g. a fluidic pump, or by any other type of actuator, all typically used in such a microfluidic device and/or (ii) can be at least partly deflected by an internal mechanical force caused by the motion of a fluid or droplet containing magnetic particles in the microfluidic transition path. This internal motion may be initiated by a magnetic field generated by said external magnetic driver to at least a part of these magnetic particles.

Such a flexible membrane may be made from a thin foil.

Such a thin foil may be for example made of polymer, such as e.g. a polypropylene, having a thickness about or less than 100 μm. For example, for such a foil, 4-40 μm deflection per mm a pressure between 0.1 mBar and 200 mBar, preferably between 0.1 mBar to 20 mBar, or between 0.1 mBar and 10 mBar, or between 0.1 mBar and 5 mBar (the latter especially relevant in case (ii) above-mentioned apply) can be applied externally onto the membrane (case (i) above-mentioned) and/or by the fluidic pressure inside the transition path due to magnetic particles actuation/motion (case (ii) above-mentioned).

In this application, the term “face” is used to define a spatial relationship between an item, and a magnetic driver. A magnetic driver generates a magnetic field which will reach a maximum value at a determinate surface of the item, when the magnet is at a certain orientation with respect to the item. When the magnetic flux acting on said surface of the item is above two-thirds of its full strength, the magnet is said to “face” the item.

In this application, the term “in proximity”, insofar as it relates to the distance between a flexible membrane and an external magnetic driver, will be understood to mean at a position from which a magnetic field from the external magnetic driver may still act on a droplet comprising magnetic particles included in the transition path or close to the transition path to create motion of at least a part of this droplet to the flexible membrane. Preferably, “in proximity” means a very small distance between the flexible membrane and the external magnetic driver, with respect to their respective sizes.

In this application, the term “external magnetic driver” will be understood to mean a source of a magnetic field. Therefore, such an external magnetic driver may be a permanent magnet made, for example, from a piece of neodymium or any other permanently magnetic material known to a person skilled in the art. Alternatively or in combination, the external magnetic driver may be an electromagnet. Such an electromagnet can be made with a coil of wire, for example. When a current flows through the coil of wire, there is a resultant magnetic field.

The external magnetic driver may be arranged to move relative to the micro-fluidic device, preferably in a path following the micro-fluidic transition path, such that when energized, a droplet of liquid containing magnetic particles can be dragged through the micro-fluidic transition path by magnetic forces. The external magnetic driver may, for example, be arranged on a means for moving the external magnetic driver, for example a motor or rack and pinion arrangement.

Alternatively, the external magnetic driver may, for example, be a linear phase-step motor formed from a multi-pole magnet. The currents through the multi-pole magnet can be controlled in such a way that a droplet of fluid containing a plurality of magnetic particles can be dragged over a long distance. The magnetic driver can consist of multiple elements, on one or on multiple sides of the micro-fluidic device.

Yet alternatively, the external magnetic driver may remain stationary, and the micro-fluidic device may be moved relative to the external magnetic driver.

During the following description, the term “a fluid containing a plurality of magnetic particles” is considered to mean a fluid containing a plurality of magnetic particles or magnetic beads, e.g. superparamagnetic particles. Examples of such particles are the Dynal (™) M270 particle, Dynal (™M) silane particles, or Nuclisens (™) particles. Other particles are known to the person skilled in the art. Such particles may be suspended in a fluid containing an analyte used in a micro-fluidic experiment. Of course, varying quantities of magnetic particles per unit volume may be used, and thus the term “particle load” can be used to refer to the relative number of particles per unit volume. In the context of a micro-fluidic analysis system using magnetic particles, the particles could contain ligands binding to a biochemical moiety of interest, a biomarker, a specific protein, nucleic acid, cell fragment, cell, a virus, or any combination of these.

These and other aspects of the invention will become apparent from, and elucidated with reference to the embodiments described hereinafter.

### BRIEF DESCRIPTION OF THE DRAWINGS

Exemplary embodiments of the invention will be described in the following with reference to the following drawings:

FIG. 1 illustrates an example of a micro-fluidic device.

FIG. 2 illustrates the micro-fluidic device in operation.

FIG. 3 illustrates the operation of a micro-fluidic device.

FIG. 4 illustrates an example of a micro-fluidic device in operation.

FIG. 5 illustrates alternative embodiments of a micro-fluidic device.

FIG. 6 illustrates another embodiment of the micro-fluidic device.

FIG. 7 illustrates an example of a micro-fluidic device.

FIG. 8 illustrates an example of a micro-fluidic system.

FIG. 9 illustrates the operation of a micro-fluidic system.

FIG. 10 illustrates an example of a testing device.

FIG. 11 illustrates a method.

FIG. 12 illustrates an experimental arrangement of a micro-fluidic device according to a specific example.

FIG. 13 illustrates foil bending as a function of the amount of magnetic particles according to a specific example.

FIG. 14 illustrates further experimental results according to a specific example.

### DETAILED DESCRIPTION OF EMBODIMENTS

According to the invention, a micro-fluidic device 10 for fluidic sample analysis is provided. The micro-fluidic device 10 comprises:
a rigid base structure 14,
a micro-fluidic transition path 16, and
a flexible membrane 18 at least partially covering the micro-fluidic transition path, wherein the micro-fluidic transition path is provided by with the rigid base structure.

In addition, the flexible membrane is adapted be placed in proximity to an external magnetic driver. The external magnetic driver may be a permanent magnet arranged so as to be movable with respect to the valve, for example, by mounting the magnet and/or the device with the micro-fluidic device on a movable support. Alternatively, the external magnetic driver may contain electromagnets or a multi-pole magnet. Multi-pole magnet coils may be controlled to implement a linear phase-step motor which drags the beads over long distances through the micro-fluidic device 10.

The micro-fluidic device 10 is configured so that when a fluid containing a plurality of magnetic particles approaches the micro-fluidic transition path and a magnetic force is applied by the external magnetic driver near the flexible membrane, the magnetic particles are attracted towards the external magnetic driver. The flexible membrane is deflectable in the direction of the external magnetic driver, and the magnetic particles move towards the external magnetic driver with the fluid.

It is envisaged that the micro-fluidic device 10 will be placed in proximity to, or facing, an external magnetic driver, which could, for example, be in a micro-fluidic controller (so-called micro-fluidic device reader in the following), or testing device.

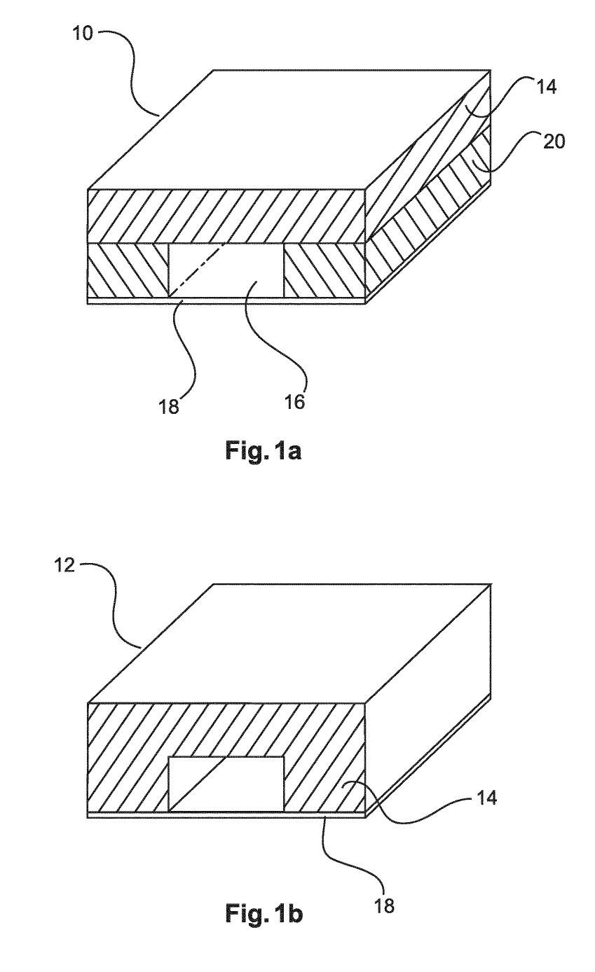

FIG. 1A illustrates the micro-fluidic device 10. In FIG. 1A, there is shown a rigid base structure 14. The rigid base structure 14 may be formed from a plastic material, glass, silicon, or any other substantially rigid material. It will be appreciated that the rigid base structure may be formed from a unitary piece of material, as shown in FIG. 1B, or it may be formed from a rigid base material 14 with additional rigid members 20 affixed on top of the rigid base material.

If the rigid base structure is made from a unitary piece of material, the micro-fluidic transition path 16 may be formed by milling, etching, or other known methods of material removal, material forming, or material addition.

The rigid base structure 14 provides a means for handling and accurate mechanical alignment of the micro-fluidic device 10.

The flexible membrane 18 is attached to the rigid base structure 14. The flexible membrane 18 may be glued to the rigid base structure 14, or attached using any other suitable method of attachment.

According to an embodiment of the invention, it will be appreciated that the flexible membrane 18 is adapted to face an external magnet.

In an embodiment of the invention, it will be appreciated that the flexible membrane may extend only over a portion of the micro-fluidic transition path 16, and the remainder of the micro-fluidic transition path may be formed by a rigid base structure 14, which is not flexible in comparison to the flexible membrane 18.

Typical materials that could be used for the rigid base structure 14 are glass, possibly with a micro-fluidic channel defined by a hydrophilized section, silicon, plastic, or other relatively rigid materials.

Typical materials used for the flexible membrane 18 could, for example, be a thin organic or inorganic material, a thin metallic foil, a thin plastic sheet, thin-film Teflon (™), or a combination thereof.

The micro-fluidic device 10 can be arranged between two micro-fluidic reaction chambers. In operation, a magnetic fluid is placed in a first chamber. An external magnetic driver located near the flexible membrane is then activated to attract the fluid containing a plurality of magnetic particles towards the entrance of the micro-fluidic device 10. Then, the external magnetic driver may move the magnetic field applied along the length of the micro-fluidic transition path through the micro-fluidic device 10. The magnetic force will attract the fluid containing the plurality of magnetic particles into and through the micro-fluidic transition path as the magnetic field moves with the motion of the external magnetic driver (or pole of a multi-pole magnet, if, for example, a synchronous linear motor is used). Eventually, the fluid containing the plurality of magnetic particles will be deposited in a second reaction chamber at the other side of the micro-fluidic transition path.

Preferably, the thickness of the flexible membrane is equal to or lower than 100 micrometres. This improves the proximity between the bottom of the micro-fluidic device and the external magnetic driver to less than 100 micrometres, unlike the case where a rigid base is used in the micro-fluidic transition channel.

Owing to the thinness of the flexible membrane, the absolute distance between the nearest magnetic particles in the fluid and the external magnetic driver can be reduced. Thus, there is a significant improvement in proximity between magnetic particles in the fluid and the external magnetic driver, which in turn increases both the magnetic field gradient as well as the absolute magnetic field strength that the magnetic particles experience. The higher gradient and higher field strength increases the attraction forces between the magnetic particles. This results in a higher packing density between the magnetic particles in the fluid, and a higher attraction strength when the external magnetic driver is moved.

The flexible membrane 18 is designed at least using knowledge of the dimensions of the valve, and knowledge of the Young's Modulus of the material used to provide the flexible membrane. These parameters are selected so that when the flexible membrane supports a droplet of fluid containing a plurality of magnetic particles, there is a deflectation (deflection) of the flexible membrane towards the external magnetic driver when an external magnetic field is applied.

This flexibility of the flexible membrane lowers the requirements on mechanical alignment of the rigid base member, compared to a micro-fluidic valve without a flexible membrane. This is because the deflectation of the flexible membrane will ensure an adaptable proximity between the magnetic driver and the particles in the magnetic fluid with a lower dependence on the initial proximity of the rigid base structure 14. The design of micro-fluidic equipment requires strict mechanical tolerances to be observed.

Advantageously, the use of a flexible membrane in the micro-fluidic device 10 relaxes the design tolerances of a micro-fluidic device, or a reader, to be used, thus allowing less onerous production processes to be employed.

In an example, the flexible bottom surface is connected to the rigid base structure by the wall elements, by an adhesive compound, or by thermal fusion of the flexible membrane and the rigid base structure, although it will be understood be the skilled person that any suitable attachment technique may be used.

The diameter of the magnetic particles used in the fluid as applied to the magnet system or micro-fluidic device 10 according to the present invention lies in the ranges of: between 3 nanometres and 15,000 nanometres, preferably between 10 nanometres and 5,000 nanometres, and more preferably still between 15 nanometres and 3,000 nanometres.

The magnetic particles as applied to the present invention can be used as carriers for biological targets. The magnetic particles can be coated with a biologically-active layer in order to bind other substances. Alternatively, the magnetic particles themselves can be utilized for detection purposes. Detection can be based on any property of the particles such as the magneto-resistive effect, the Hall effect, or through optical methods. The magnetic particles may be equipped with a fluorescent dye, allowing optical methods such as fluorescence, chemiluminescence, absorption, or scattering.

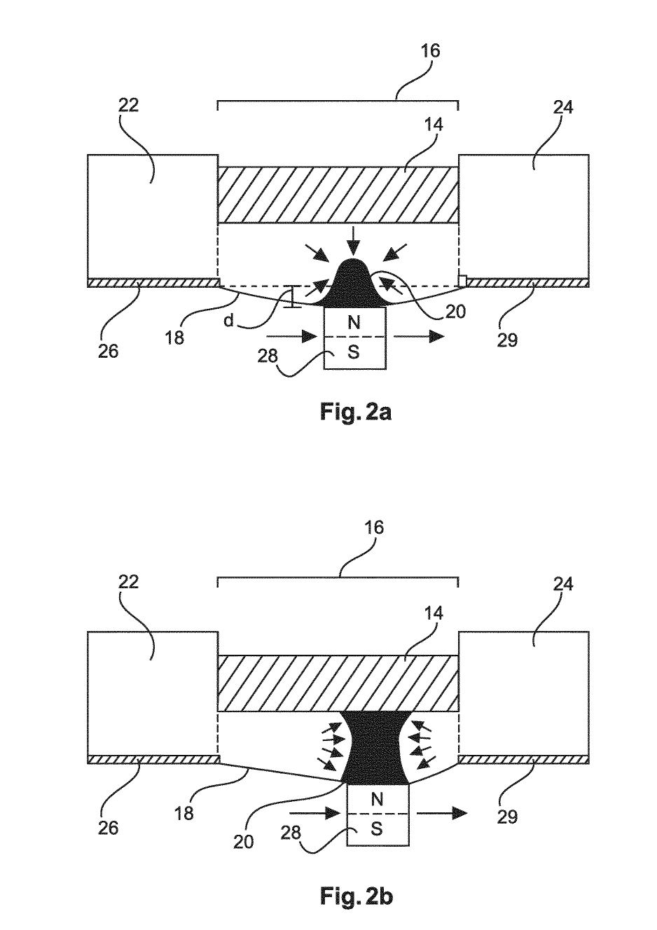

FIG. 2A shows the micro-fluidic device 10 in operation. The fluid containing a plurality of magnetic particles 20 is shown in transition between first reaction chamber 22 and second reaction chamber 24. It will, of course, be appreciated that more than two chambers may be provided with a plurality of micro-fluidic devices 10 connecting them.

In an exemplary embodiment, the bottom surfaces 26 and 29 of the first and second reaction chambers, and a top surface 14 of the micro-fluidic transition path comprise a rigid surface.

It will be seen that the external magnetic driver 28 shown in FIG. 2 is in the middle of a transition between the first 22 and second 29 chambers. The flexible membrane 18 is deflected towards the external magnetic driver 28. This is because the external magnetic driver 28 exerts a magnetic force on the magnetic particles inside the fluid 20. The force of the magnetic particles attracted the external magnetic driver 28 is incident on the flexible membrane 18. Therefore, the flexible membrane deflects a distance, d, towards the external magnetic driver 28.

As represented by the small, inwardly-pointing arrows surrounding the fluid containing a plurality of magnetic particles 20 in FIG. 2A, the increased proximity to the external magnetic driver 28 improves the packing density of the magnetic particles inside the fluid, giving the fluid drop greater integrity against the friction forces exerted by the flexible membrane.

In the case of FIG. 2A, the dimensions of the micro-fluidic device 10 are such that the droplet containing a plurality of magnetic particles 20 is not in contact with the rigid top of the micro-fluidic transition path. In fact, in an alterative embodiment, no upper surface of the transition path is required. In the case of FIG. 2A, there is reduced friction from the top surface, and reduced capillary forces, owing to the fact that the fluid is less in contact with the rigid top of the micro-fluidic transition path.

FIG. 2B also shows a micro-fluidic device 10 connecting a first chamber 22 to a second chamber 24. In this case, the dimensions of the micro-fluidic transition path 16 are configured so that the droplet of fluid containing a plurality of magnetic particles 20 does not lose contact with the rigid base structure 14. In this case, the droplet 20 will experience more friction, and capillary forces will be present. The external magnetic driver 28 still attracts the magnetic particles inside the magnetic fluid. Therefore, the flexible membrane 28 is deflected (deflected) in the direction of the external magnetic driver 28.

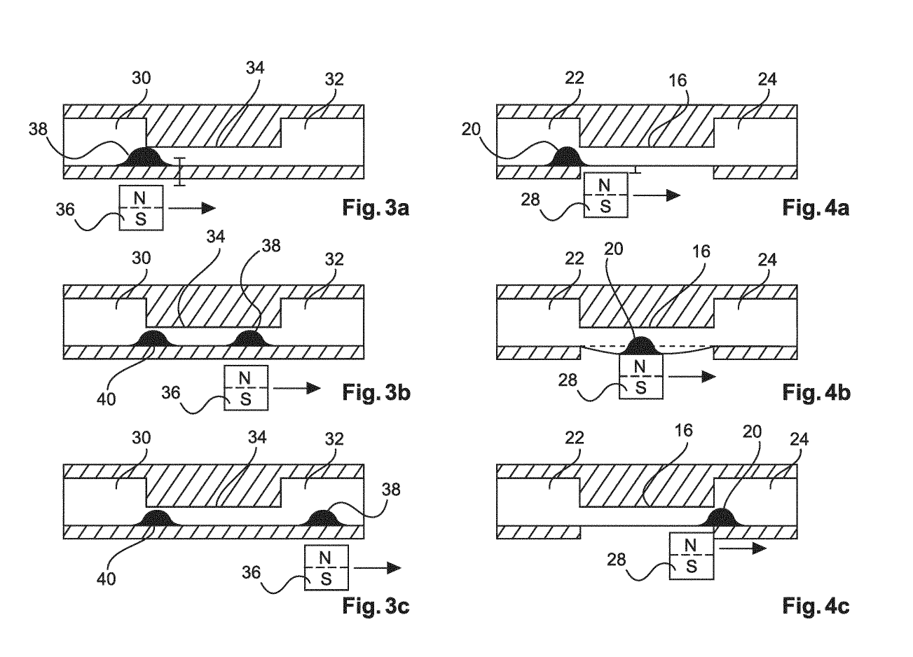

FIG. 3 demonstrates a problem with a prior art micro-fluidic device 10. In FIG. 3A, a micro-fluidic device with a rigid bottom is provided, which transitions between a first chamber 30 and a second chamber 32. A micro-fluidic transition path 34 connects the two chambers. The external magnetic driver 36 attracts the fluid containing a plurality of magnetic particles to the exit of the first chamber 30. Then, as shown in FIG. 3B, the external magnetic driver, or the active point of a multipole magnet, is moved along the outside of the micro-fluidic channel 34. Because the external magnetic driver, is a relatively large distance away from the bottom of the micro-fluidic channel 34, owing to the rigid surface in-between the channel and the external magnetic driver, it will be seen that the fluid containing a plurality of magnetic particles separates into two droplets during a transition between the chambers. The first droplet 38 continues to be transported through the micro-fluidic channel. A second droplet 40 remains at the entrance of the micro-fluidic channel 34. Such “cloud splitting” (droplet-splitting) occurs owing to the complex force-balance relationship between a droplet and the surfaces in a magneto-capillary valve.

Viscous friction results between the particles inside the fluid droplet, or surface friction between the particles and the micro-fluidic device surface, or contact line friction between the fluid and micro-fluidic channel's surface. Internal friction results from friction between particles inside the droplets.

Capillary forces can be characterized in several phases throughout the inter-chamber transport.

Finally, the particle load (the number of particles in a droplet inside the fluid) is a useful parameter for determining the magnetic force, the capillary force, and the friction force.

A reduction of the number of magnetic beads in the fluid can also reduce the friction forces, and help to prevent cloud splitting. The use of a flexible (deflectable) member in a micro-fluidic valve allows a smaller number of magnetic particles to cross the magneto-fluidic valve, owing to the greater magnetic field. In practice, measured results show that a reduction by four times in the number of beads is possible (with a flexible member of 0.03 mm thickness) compared to when a rigid member of 0.5 mm is used.

It is known that magnetic force between a droplet and an external magnetic driver increases with the particle load of the droplet. To a first-order approximation, the increase is linear with the amount of magnetic particles. It is known, though, that with increasing droplet diameter, the particles are distributed over a wider lateral distance. This lowers the increase nonlinearly. Thus, the forces required to “wet” the surface of a fluidic-valve arrangement increase with the increasing droplet diameter. The increasing droplet diameter also increases the viscous friction that the droplet experiences. The surface friction increases due to the increase of the magnetic normal force. These effects all make the problem of droplet splitting shown in seen in FIG. 3 more likely to occur.

As shown in FIGS. 4A-C, during the transition between the first chamber 22 and the second chamber 24, the droplet of fluid containing a plurality of magnetic particles 20 is positioned at the entry of the micro-fluidic transition path 16. The external magnetic driver 28 is shown moving along the micro-fluidic transition path in FIG. 4B. The droplet of fluid containing magnetic particles is attracted towards the external magnetic driver, thus deflecting the flexible membrane 18 towards the magnet. The initial increase in proximity is caused by the inherent thinness of the flexible membrane. The additional proximity is caused by the deflection of the flexible membrane towards the external magnetic source 28. This increases the magnetic force on the droplet, thus advantageously allowing a greater packing density of magnetic particles in the droplet. Therefore, as shown in FIG. 4C, the droplet of fluid containing a plurality of magnetic particles is transported between chamber 22 and chamber 24 without dividing into several droplets. In other words, cloud-splitting does not occur.

According to an embodiment of the invention, a micro-fluidic device 10 is provided which comprises a fluid 20 containing a plurality of magnetic particles. The fluid moves through the micro-fluidic channel 16 when an external magnetic driver 28 applies a magnetic force in proximity to the magnetic channel. Advantageously, if the micro-fluidic device 10 is provided already including a fluid comprising magnetic particles, the user of the micro-fluidic device 10 does not need to provide the fluid containing magnetic particles externally.

According to an embodiment, a micro-fluidic device 10 is provided in which the flexible membrane 18 has a thickness of 100 micrometres, preferably less than 80 micrometres, more preferably less than 60 micrometres, and most preferably less than 40 micrometres. The thin flexible membrane lowers the absolute distance between the nearest magnetic particles and the magnet, thus further increasing the magnetic force.

According to an embodiment, a micro-fluidic device 10 is provided where the flexible membrane comprises a roughened surface, at least on a side of the micro-fluidic transition path facing the rigid base structure.

It will be appreciated that a rougher flexible membrane will apply more friction to the droplet of fluid containing a plurality of magnetic particles. As will be gathered from the previous discussion on the force balance involving magnetic forces, friction forces, and capillary forces inside the micro-fluidic transition path, such a roughness could disadvantageously increase the friction in the micro-fluidic transition path. This could allow droplets inside the micro-fluidic transition path to split as they transition through the path.

Advantageously, therefore, because the flexible membrane 18 may deflect more closely to the external magnetic driver, the additional friction implicit in the use of a roughened surface is cancelled-out by the higher magnetic force incident on the droplet. Therefore, a foil with a rougher surface can be used in the construction of the micro-fluidic device 10, which may be less expensive.

Alternatively, a roughened surface may exhibit a higher bending flexibility, for example, because the surface is corrugated. A corrugated surface is mechanically more easily deflected.

Quantities typically used to characterize surface roughness are the arithmetic mean value, Rₐ, the quadratic mean, R_{q}, and the maximum roughness height, Rₜ, as would be known to the skilled person.

In the micro-fluidic devices discussed herein, an Rₐ of up to 0.3 micrometres can be tolerated, with an Rₜ of up to 20 micrometres when a flexible membrane of 0.03 millimetre thickness is used. This differs from the case where the bottom of the micro-fluidic transition channel is made from a rigid and thick bottom surface. A glass plate, as used conventionally, may have a thickness of 1.1 mm. For acceptable could-splitting performance, the requirement of surface roughness of such a glass plate has been found to be as low as an Rₐ of only 10 nanometres, and Rₜ of 0.3 micrometres. This is because of the reduced magnetic force caused by the increased separation of the magnetic fluid and the external magnetic driver, caused by the thickness of the glass plate. The Rₐ and Rₜ values were determined over an area with the dimensions 0.5 by 0.5 millimetres. Thus, it can be seen that providing a flexible membrane in the micro-fluidic transition channel advantageously allows a relaxation of the roughness requirements of the surface of the micro-fluidic channel.

Seen another way, when a thin foil surface is used, the same magnetic field incident on the micro-fluidic channel can be achieved using lower energy at the external magnetic driver 28, because the micro-fluidic channel is closer to the external magnetic driver. This is an important consideration if electro-magnets, or multi-pole magnets are used, and the device reader is a hand-held, and possible battery-powered device. If a lower magnetic field strength is needed, the battery will last for longer.

According to an embodiment, a micro-fluidic device 10 is provided which further comprises a flexible membrane deflecter 42 or 46. The flexible membrane may be mechanically deflected by the flexible membrane deflecter, so to contact the surface of the rigid member, thereby forming a flow-constricted position for peristaltic fluid transfer. An example of a mechanical surface deflecter may be a mechanical element contacting the flexible membrane and driven by a micro-miniature or MEMS servo element, although other implementations are possible.

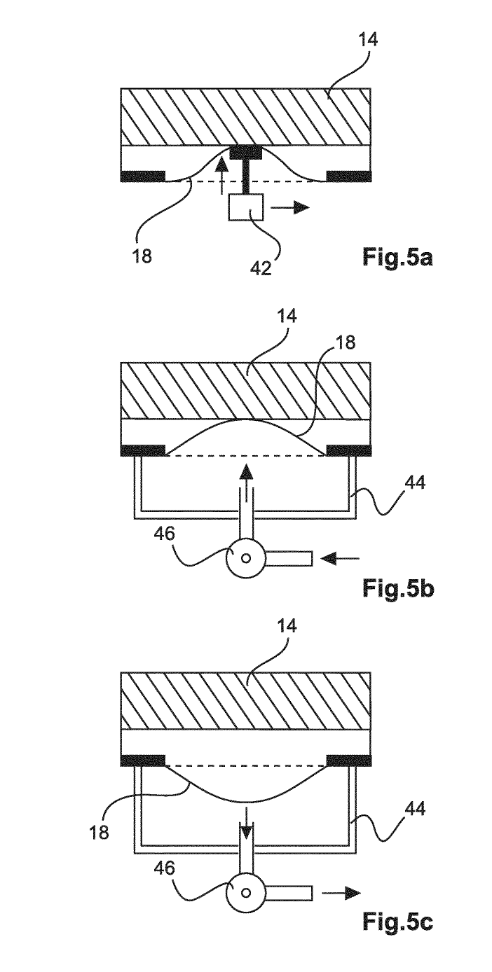

FIG. 5A illustrates such an arrangement. A flexible membrane deflecter 42 is arranged underneath the micro-fluidic transition channel 16. The flexible membrane deflecter can be moved upwards, so as to restrict, or to close the micro-fluidic transition channel. In addition, the flexible membrane deflecting means 42 may be moved along the micro-fluidic transition channel so as to move the location of the blockage. In this way, a peristaltic transport mechanism is provided.

Additional examples of flexible membrane deflecters are shown in FIGS. 5B and 5C. In FIG. 5B, a sealed, fluid-tight chamber 44 surrounds the flexible membrane 18, which can be said to form a diaphragm. A fluid such as air or another liquid may be forced into the chamber 44 by the pump 46. A resulting deflectation in the flexible membrane 18 occurs towards the rigid member 14 forming the micro-fluidic transition path. In this way, the two sides of the micro-fluidic transition path are sealed from each other. Alternatively, the application of an intermediate fluid pressure may simply restrict the flow through the micro-fluidic transition path 16, causing the micro-fluidic transition path to act as a flow resistance.

In the embodiment of FIG. 5C, a chamber 44 is again arranged around, and in sealable contact with, the flexible membrane 18 functioning as a diaphragm. However, in this embodiment, fluid such as air, or a liquid, may be drawn out of the chamber 44 using the pump 46. Therefore, the flexible membrane 18 is sucked downwards, away from the opposite surface of the rigid member 14. Such an arrangement may be useful for drawing a droplet into the micro-fluidic transition path. It will be appreciated that the embodiments shown FIGS. 5A, 5B, and 5C may be used alone, or in combination, with the external magnetic driver as described previously.

In an embodiment, the external magnetic driver may also be a mechanical deflecting element.

According to an embodiment of the invention, a micro-fluidic device 10 is provided that is configured to form a local under-pressure to bring fluid in the micro-fluidic transition path 16 into motion. Therefore, the defined volume of the under-pressure which is provided by tuning the deflectation length and surface prevents fluid-flow occurring further than that designed. This is also referred to as a fluidic-stop.

According to an example, a micro-fluidic device 10 is provided that further comprises a heater 48. When the heater is activated, a lateral temperature gradient is applied by the heater to the micro-fluidic channel 16. This enables a thermal processing operation to be performed inside the micro-fluidic channel.

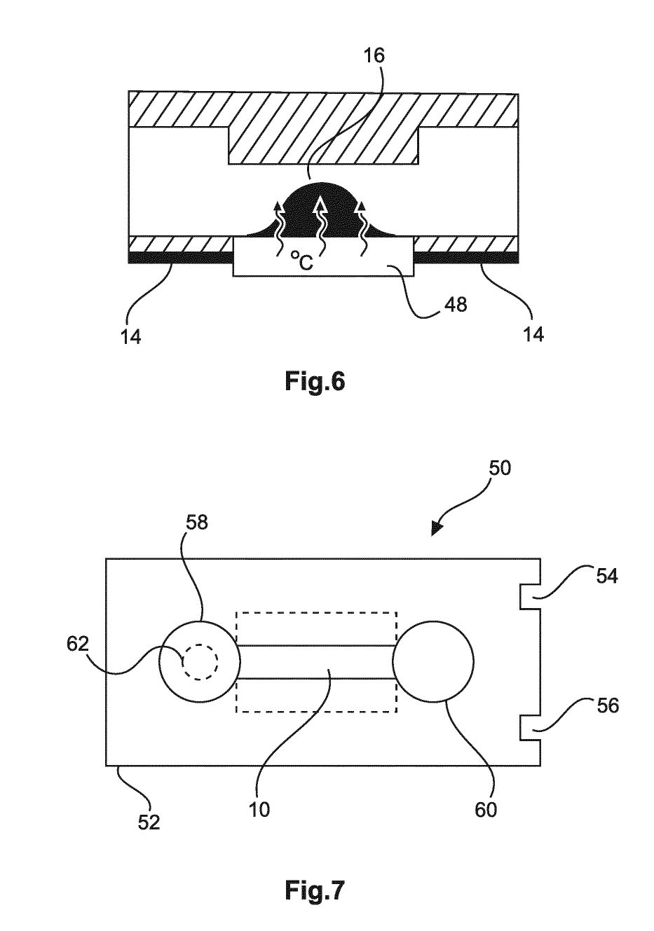

FIG. 6 shows an example of such a heater. Furthermore, with a lateral temperature gradient in combination with convection, concentration of solutes within a droplet can be achieved.

It will be appreciated that the lateral temperature gradient can be used to tune the solubility of solutes like RNA, DNA, and proteins, although there are many other uses.

According to an example, the temperature gradient in the micro-fluidic transition path may have a magnitude of greater than or equal to 70° C. per millimetre.

According to an example, the heater may be arranged under a specific portion of the valve-arrangement.

According to an exemplary embodiment, a micro-fluidic device 10 is provided where the flexible membrane 18 is, upon actuation by an external driver, deflectable by forces selected from the group of: mechanical contact forces, pressure forces, vacuum forces, acoustic or sonic forces, capillary forces, or electromagnetic field forces.

According to an example, a micro-fluidic device 10 is provided where the maximum force exerted on the flexible membrane 18 is lower than a rupture force of the flexible membrane. Therefore, there is no risk of breakage of the flexible membrane caused by the increased magnetic force.

According to an example, a cartridge 50 is provided, comprising a cartridge housing 52 with a cartridge holding means 54, 56. The cartridge also comprises at least two fluidic chambers 58 and 60. Furthermore, the cartridge comprises a micro-fluidic device 10 as discussed previously. The cartridge holding means 54 and 56 can be mounted in a cartridge reader device, and the at least two fluidic chambers are connected by the micro-fluidic device 10.

Therefore, a cartridge having a micro-fluidic device 10 with the advantageous behaviour described previously is discussed. The flexible membrane of the micro-fluidic device 10 may form the bottom surface of the cartridge. Therefore, when the cartridge is inserted into a cartridge reader, the bottom surface of the cartridge is in close proximity to the cartridge reader.

The cartridge comprises a fluid entry hole 62 which allows a sample of reagents to be applied to the cartridge before a measurement.

According to an exemplary embodiment, the micro-fluidic device 10 is provided with a dried reagent containing magnetic particles. Therefore, in use, fluid is added to the magnetic reagent so as to form a fluid containing magnetic particles. This allows micro-fluidic devices containing magnetic particles to be stored in a dry state for a long time.

According to the invention, a micro-fluidic system 64 is provided. The system comprises a micro-fluidic device reader 66. The micro-fluidic device reader comprises a micro-fluidic device placement area 68 compatible with a micro-fluidic device, such as a cartridge, as previously described. An external magnetic driver is placed in proximity to the micro-fluidic device placement area. The external magnetic driver is configured to apply a magnetic field to the micro-fluidic device placement area, and is also able to move around underneath the micro-fluidic device, to manipulate droplets containing magnetic particles contained inside. In addition, the micro-fluidic system comprises a micro-fluidic device 52 according to the previous description.

Alternatively, the external magnetic driver may be stationary, and the micro-fluidic system may be configured to move the micro-fluidic device (cartridge) in the micro-fluidic device placement area, to achieve the necessary relative movement to move a droplet containing magnetic particles.

In use, a fluidic medium is introduced into the micro-fluidic device, and the micro-fluidic device is then secured in the micro-fluidic device placement area 68 of the micro-fluidic device reader. When the plurality of magnetic particles approaches a micro-fluidic transition path 16 of the micro-fluidic device 10, and a magnetic force is applied by the external magnetic driver at a flexible membrane 18 of the micro-fluidic device, the magnetic particles are attracted towards the external magnetic driver. The flexible membrane is deflectable at least in the direction of the external magnetic driver, and the magnetic particles to move towards the external magnetic driver with the fluid.

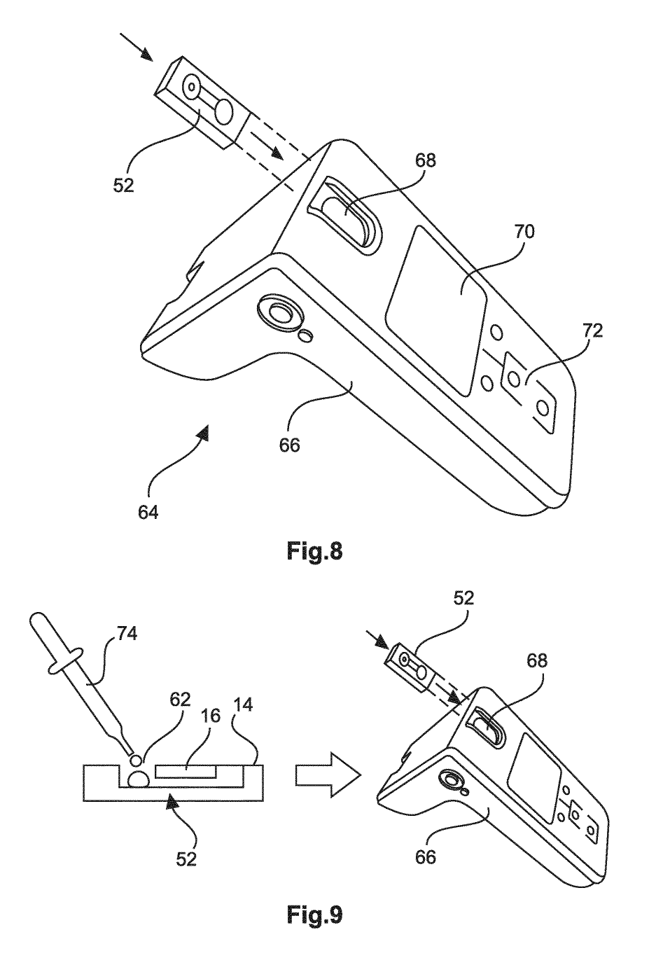

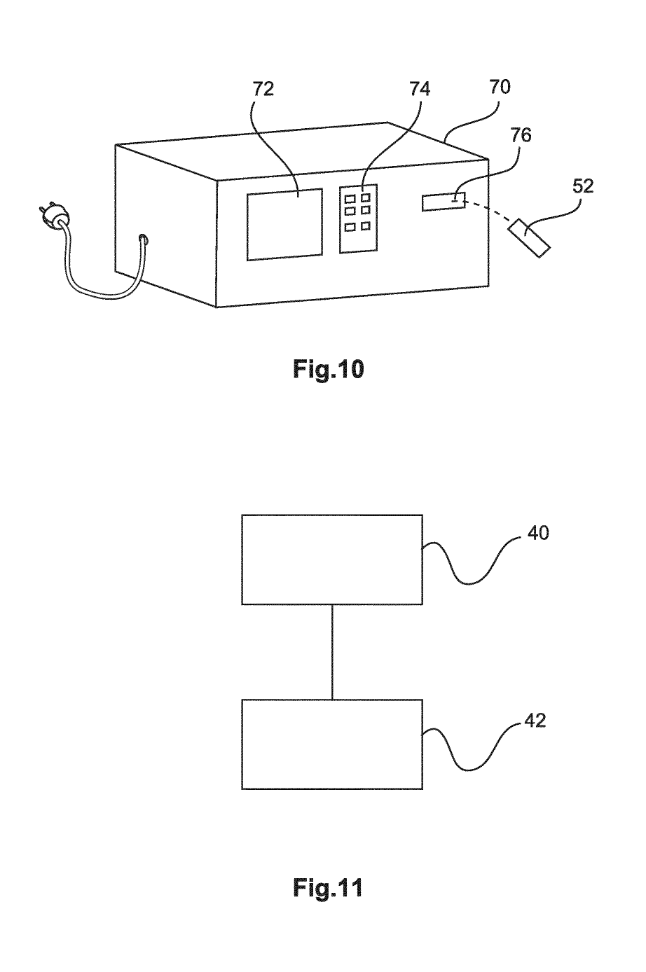

FIG. 8 illustrates such a magneto-fluidic system 64. The reader advantageously allows measurements of medical conditions, for example, to be made closely to the point-of-care. The reader 66 comprises a display 70 and a control panel 72. When a micro-fluidic device 52 is placed into the micro-fluidic device (cartridge) placement area 68, the reader 66 performs a number of measurements and operations on the micro-fluidic device 52, potentially involving the use of a moving external magnetic driver to manipulate fluids containing magnetic particles in the micro-fluidic device. Then, results are read from the cartridge into the reader, and the results may be displayed directly on the screen 70 of the reader, the results may be stored for further use, or the results may be transmitted. It is noted that such use statements are not restrictive, and other uses of the information read from the micro-fluidic device are possible.

Advantageously, the flexible membrane of the micro-fluidic channel 16 comprised within the micro-fluidic device (cartridge) 52 means that the micro-fluidic device can be placed much more closely to the external magnetic driver contained in the handset 66. The micro-fluidic device 52 allows much more effective control over the movement of a fluid containing magnetic particles in the micro-fluidic channel. This results in a more efficient use of analytes and reagents, and more reliable functioning of the micro-fluidic device, which leads to better quality results.

FIG. 9 shows the magneto-fluidic system in use. A micro-fluidic device 52 has a fluid applied, for example, with a pipette 74. The fluid may be, for example, blood from a blood test. The fluid is added using the pipette to the micro-fluidic device fluid entry area 62.

In an alternative embodiment, extra fluid, such as water, may be added before the reagent is added. This, for example, allows the wetting of a dried reagent containing a plurality of magnetic particles. When the fluid has been applied to the micro-fluidic device 52, the micro-fluidic device 52 is then inserted into the handheld reader 66 in the micro-fluidic device (cartridge) placement area 68. Then, analysis operations can begin.

According to an exemplary embodiment, a magneto-fluidic system 64 is provided that further comprises a micro-fluidic device 10 containing a fluid 62 containing a plurality of magnetic particles, wherein the fluid is configured to move through the micro-fluidic channel 16 of the micro-fluidic device 10 when the external magnetic driver 28 is placed in proximity to the micro-fluidic channel.

According to an embodiment of the invention, a magneto-fluidic system 64 is provided, where the micro-fluidic device reader 66 further comprises a camera configured to image the micro-fluidic device placement area 68; and wherein the flexible membrane 18 of the micro-fluidic device is transparent; and wherein, in use, the micro-fluidic device is placed in the micro-fluidic device reader, and the camera allows magnetic particles to be imaged.

The flexible membrane enables the micro-fluidic device to be placed much more closely to the external magnetic driver. Therefore, additional focussing optics used to image a reaction taking place inside the micro-fluidic device are not needed. This advantageously reduces the cost of a handheld reader.

Of course, the micro-fluidic device which is insertable into the magneto-fluidic system or testing device discussed above may take the form of a cartridge, as previously discussed in the example above.

According to an exemplary embodiment, the micro-fluidic device placement area 68 further comprises a protective layer arranged to sealably cover the external magnetic driver 28, thereby to protect the inside of the magneto-fluidic system from fluid ingress.

As a result of the addition of the protective layer, the minimum separation distance between a top surface of the reader comprising the external magnet, and the bottom of the flexible layer of the micro-fluidic device, is equal to the thickness of the protective layer.

According to an exemplary embodiment, a testing device is provided, comprising:
at least two fluidic chambers;
a micro-fluidic device 10 as described previously; and
a magnetic particle transferrer located underneath the micro-fluidic device 10.

The at least two fluidic chambers are connected by the micro-fluidic device 10, and, in use, the magnetic particle transferrer moves a quantity of fluid from a first to a second of the at least two fluidic chambers.

In this way, a testing device, as may be useful in a laboratory, for example, is provided which can accept the micro-fluidic devices 10, in cartridge form, for example, and process micro-fluidic droplets more efficiently.

According to the invention, a method of controlling fluid flow comprising the steps of:
a) inserting fluid containing a plurality of magnetic particles into a micro-fluidic device 10 with a flexible wall; and
b) applying an external magnetic field to the micro-fluidic device 10, thus causing the force against the flexible membrane, so deflecting the flexible membrane in the direction of the external magnetic driver, and causing the magnetic particles to move towards the external magnetic driver with the fluid.

Accordingly therefore, a method is provided of transferring a fluid containing magnetic particles between a first and a second chamber, allowing improved proximity between the droplets of fluid and the external magnetic driver. This, in turn, allows an improvement in the magnetic field gradient and the absolute magnetic field that the magnetic particles experience. This increases the attraction force between the particles and the magnet, resulting in a higher packing density and higher attraction strength, even when the magnet is moved. This maintains the integrity of the droplet of fluid, preventing droplet splitting.

According an aspect of the invention, a kit of parts for fluidic sample analysis is provided, comprising:
a micro-fluidic device as previously described;
a cartridge comprising a fluid;
wherein the cartridge is configured to apply the fluid reagent to the a micro-fluidic transition path of the micro-fluidic device.

Therefore, a fluid for use with the micro-fluidic device may be more easily provided for use with the micro-fluidic device. In an example, the cartridge is a plastic, disposable ampoule, with a “one-time” use tearable stopper atop an injection means, for example, a nozzle, sized to inject the fluid reagent into the micro-fluidic transition path of the micro-fluidic device. The cartridge may be made from polyethylene, polycarbonate, polypropylene, PET, or the like.

The fluid can comprise water, or a reagent suitable for use in a magneto-fluidic assay, or buffer salts dissolved in water. In an example, the fluid reagent may also comprise magnetic particles.

In use, the tearable stopper is removed from the ampoule. The reagent inside is applied to an area of a micro-fluidic device containing sample material. The micro-fluidic device can then be applied to a reader for analysis of a sample.

### Specific Example

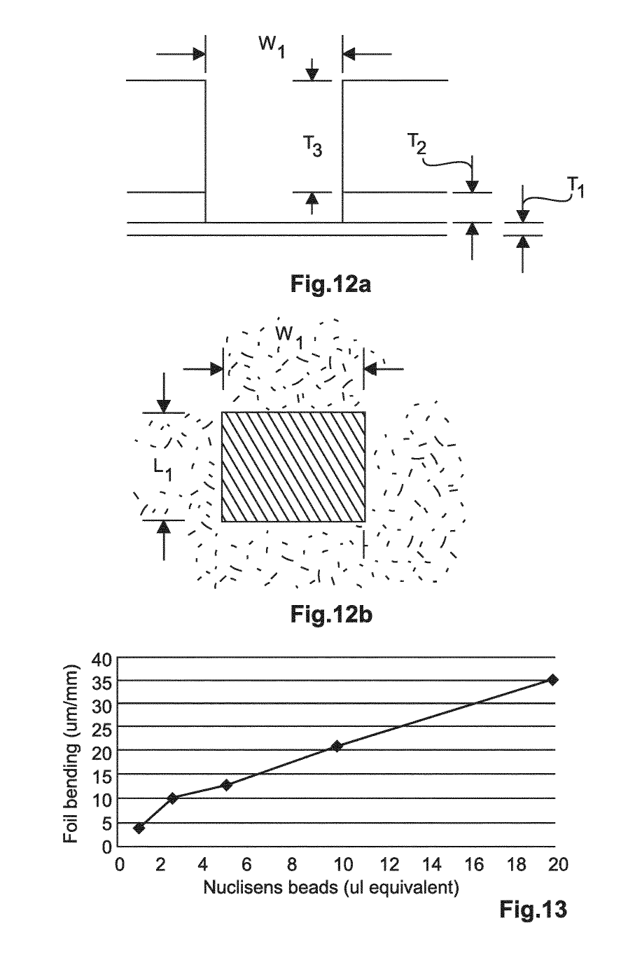

There follows a discussion of a specific example of a micro-fluidic valve, with measurements determined experimentally, to demonstrate the advantageous effects discussed above. The valve is illustrated in FIGS. 12a) and b). A valve was constructed using, as the rigid member 14, pressure-sensitive adhesive tape “1505P” (0.18 mm iso 0.22 mm) supplied by the Nitto Denko corporation (™), illustrated in FIG. 12a) by the layer T₂. A micro-fluidic transition channel was formed in the rigid member by a laser machining method. Then, the rigid member was reinforced at one side using a poly-methyl methacrylate (PMMA) plate, illustrated in FIG. 12a) by layer T₃, with the corresponding structure to that in the adhesive tape layer laser-machined out of the plate.

The flexible membrane was made using biaxially-stretched polypropylene (PP) foil having a thickness of 30 micrometres +/−3 micrometres. This foil has a Young's modulus of 1.5 GPa, and a surface roughness defined with an Rₐ value of 0.3 micrometres and Rₜ of 15 micrometres. In FIG. 12a), this is denoted layer T₁.

The flexible membrane was attached to the rigid assembly using the adhesive properties of the pressure-sensitive adhesive tape.

With reference to the dimension markings in FIGS. 12a) and b), the dimensions of the exemplary valve arrangement were W₁=L₁=4 millimetres, T₁=0.03 millimetres, T₂=0.22 millimetres, T₃=3 millimetres.

In a resting state, the flexible member was substantially flat, and parallel to the upper surface of the rigid member.

The magnetic particles used in this example are superparamagnetic Nuclisens (™) particles.

A droplet with a volume of approximately 3 microlitres was introduced onto the flexible member. The particle contained approximately 18 percent magnetic particles by volume.

An external magnetic source was positioned underneath, and on the opposite side of, the flexible member to where the droplet was located. The magnet was a permanent magnet of 4 millimetres diameter, 5 millimetres length, and having a remanescent magnetisation of 1.2 Tesla. This magnet applied a flux strength of 0.62 Tesla at a distance of 0.25 millimetres from the flexible membrane.

Measurement of the deflection of the flexible membrane was performed using a Wyko (™) NT110 white light interferometer, having an accuracy of better than 0.1 micrometres.

The variation applied in the experiment was the volume of particles applied, in this case from 3 to 20 microlitres of particles. The deflection of the flexible member was measured over a 1 millimetre length.

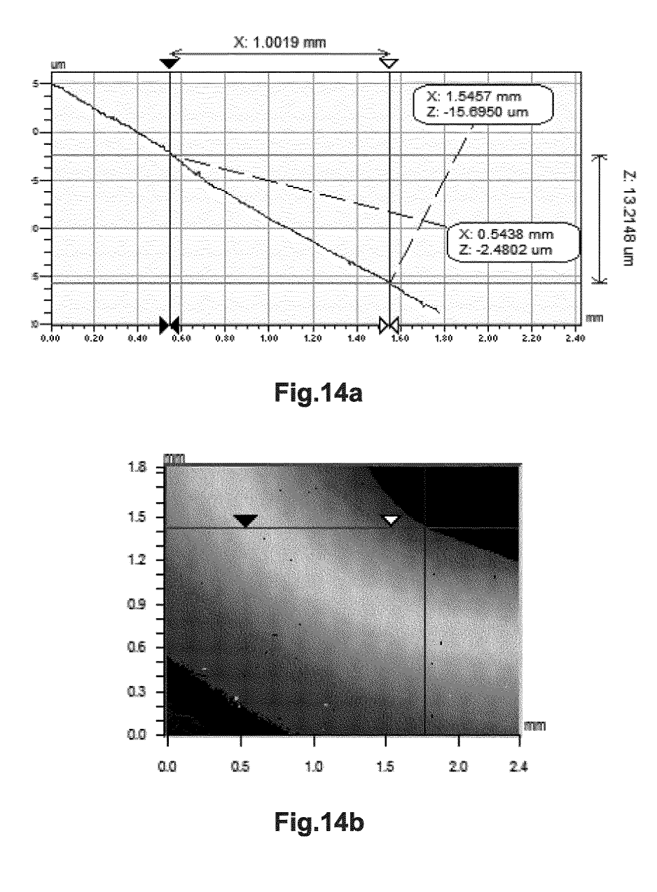

Example deflection profiles are shown in FIG. 14. From these, it can be concluded that a deflection of 0.03 millimetres per millimetre length can be achieved for a typical magnetic particle volume of 20 microlitres (18 volume by percent). In the typical distance between MCV valves of 4 millimetres, this means that the proximity to the magnet can be increased by 0.12 millimetres.

FIG. 13 illustrates foil bending as function of the amount of magnetic particles used.

FIG. 14A shows deflection measurements across two lines the foil element shown in FIG. 14B.

FIG. 14B shows the deflection profile across a foil element in 2D format. The axes in the x and y directions represent the location on a square of foil, and the intensity of the image represents the deflection of the foil in the Z axis (into and out of the page).

This specific example shows, therefore, the significant benefits which accrue when a micro-fluidic valve suitable for use with fluids containing magnetic particles is provided with a flexible membrane at least partially covering the micro-fluidic transition path.

It should to be noted that embodiments of the invention are described with reference to different subject matters. In particular, some embodiments are described with reference to method type claims whereas other embodiments are described with reference to device-type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A micro-fluidic device for fluidic sample analysis, arranged to be positioned in a micro-fluidic controller comprising an external magnetic driver, comprising:
a base structure;
a flexible membrane;
a micro-fluidic transition path limited by, at a bottom side, at least a portion of the base structure and by, at a top side, at least a portion of the flexible membrane, and extending between at least one inlet to be in communication with a first region and at least one outlet to be in communication with a second region; and
wherein the microfluidic device is adapted such that, once in position in the micro-fluidic controller, the flexible membrane is placed in proximity to said external magnetic driver;
wherein the micro-fluidic device further comprises:
a plurality of magnetic particles (i) arranged to be put in contact and included thereafter in a fluid included in the microfluidic device or (ii) already included in the fluid and the micro-fluidic device further comprises this fluid; and
wherein the micro-fluidic device is configured so that when the fluid containing the plurality of magnetic particles approaches or is within the micro-fluidic transition path and a magnetic force is applied by the external magnetic driver near the flexible membrane, at least a part of the magnetic particles are moved away from the base structure towards the flexible membrane, and wherein the flexible membrane is deflectable away from the base structure such that, when the flexible membrane is deflected, at least a part of the magnetic particles can be located, without the fluid or with a part of the fluid, beyond the in rest position of the flexible membrane; and
wherein the micro-fluidic device is arranged such that the magnetic particles can move, without fluid or with only a part of the fluid, through the micro-fluidic transition path when an external magnetic driver applies a magnetic force in proximity to the micro-fluidic transition path.

2. Micro-fluidic device of claim 1,
wherein the flexible membrane has a thickness of 100 micrometers or less.

3. Micro-fluidic device of claim 1,
wherein the flexible membrane comprises a roughened surface at least on a side of the micro-fluidic transition path facing the base structure.

4. Micro-fluidic device of claim 1, further comprising a membrane deflecter arranged to deflect the flexible membrane towards and/or away from the base structure.

5. Micro-fluidic device of claim 1,
configured to form a local under-pressure to bring fluid in the micro-fluidic transition path into motion.

6. Micro-fluidic device of claim 1,
wherein the flexible membrane is, upon actuation by an external driver, deflectable by forces selected from the group of: mechanical contact forces, pressure forces, vacuum forces, acoustic or sonic forces, capillary forces, or electromagnetic field forces.

7. Micro-fluidic device of claim 1, wherein the micro-fluidic transition path has a valve-like function between first and second regions, arranged to leave the magnetic particles, without or with a part of the fluid, to go from the first region to the second region when the flexible membrane is deflected and when the external magnetic driver is actuated.

8. Micro-fluidic device of claim 7, wherein the flexible membrane is adapted to face an external magnet.

9. Micro-fluidic device of claim 7,
wherein the magnetic particles and the flexible membrane are arranged such that the magnetic particles can be moved towards the flexible membrane and away from the base structure, thereby exerting a force onto the flexible membrane, so deflecting, with or without a part of the fluid, at least partly the flexible membrane in a direction away from the base structure and causing, at least partly, the magnetic particles to move away from the base structure.

10. A testing device comprising:
at least two fluidic elements;
the micro-fluidic device of claim 1; and
a magnetic particle transferrer located underneath the micro-fluidic device; wherein the at least two fluidic elements are connected through the micro-fluidic device via the micro-fluidic transition path, and, in use, the magnetic particle transferrer moves a quantity of magnetic particles, optionally with no fluid or with only a part of fluid, from a first to a second of the at least two fluidic elements.

11. A micro-fluidic system, comprising:
a micro-fluidic controller; comprising
a micro-fluidic device placement area compatible with a micro-fluidic device holder; and
a magnetic driver configured to apply a magnetic field to the micro-fluidic device placement area; and
a micro-fluidic device according to claim 1;
wherein, in use, a fluidic medium can be introduced into the micro-fluidic device; and
wherein, in use, the micro-fluidic device is secured in the micro-fluidic device placement area of the micro-fluidic controller; so that when the plurality of magnetic particles approaches a micro-fluidic transition path of the micro-fluidic device, and a magnetic force is applied by the magnetic driver near the flexible membrane of the micro-fluidic controller, the magnetic particles are attracted towards the magnetic driver, and the flexible membrane is deflectable in the direction of the magnetic driver, causing at least a part of the magnetic particles to move towards the external magnetic driver, optionally with a part of the fluid.

12. Micro-fluidic system of claim 11, wherein the micro-fluidic device placement area further comprises a protective layer arranged to sealably cover the external magnetic driver, thereby to protect the inside of the magneto-fluidic system from fluid ingress.

13. Micro-fluidic system of claim 11,
wherein the micro-fluidic controller further comprises: a camera configured to image the micro-fluidic device placement area; and wherein the flexible membrane of the micro-fluidic device is transparent; and wherein, in use, the micro-fluidic device is placed in the micro-fluidic controller, and the camera allows magnetic particles to be imaged.

14. A method of controlling fluid flow, comprising the steps of:
a) inserting fluid containing a plurality of magnetic particles into a micro-fluidic device or inserting fluid into a micro-fluidic device containing magnetic particles arranged to be in contact with the fluid, the micro-fluidic device comprising a plurality of magnetic particles (i) arranged to be put in contact and included thereafter in the fluid or (ii) already included in the fluid and the micro-fluidic device further comprises this fluid, and a flexible membrane covering a micro-fluidic transition path; and
b) applying a magnetic field to the micro-fluidic device so as to cause the magnetic particles to be attracted towards the flexible membrane,
c) deflecting the flexible membrane in the direction of the motion of the magnetic particles, causing at least a part of the magnetic particles to be located, with the fluid, beyond the in rest position of the flexible membrane.

15. A kit of parts for fluidic sample analysis comprising:
a micro-fluidic device as claimed in claim 1; and
a cartridge comprising a fluid;
wherein the cartridge is configured to apply the reagent to the micro-fluidic transition path of the micro fluidic device.
